(19) United States
(12) Patent Application Publication
(10) Pub. No.: US 2025/0205461 A1
(43) Pub. Date: Jun. 26, 2025

(54) APPARATUS FOR SHUNTING CEREBROSPINAL FLUID

(71) Applicant: Gary MAGRAM, Corona Del Mar, CA (US)

(72) Inventor: Gary MAGRAM, Corona Del Mar, CA (US)

(21) Appl. No.: 18/390,162

(22) Filed: Dec. 20, 2023

(51) Int. Cl. A61M 27/00; A61M 25/00

(52) U.S. Cl. CPC A61M 27/006; A61M 25/0026; A61M 25/0075; A61M 2025/0078

(57) ABSTRACT

Apparatus for shunting cerebrospinal fluid comprising either a sleeved ventricular catheter and/or an artificial dural sac (ADS). The sleeved ventricular catheter comprises an inner catheter and an outer catheter that circumscribes and is spaced apart from at least a portion of the inner catheter to form a gap. The inner catheter comprises a plurality of apertures through which CSF may enter the inner catheter. The ADS comprises a flexible wall defining a volume where CSF accumulates. The ADS is coupled to the ventricular catheter via an ADS catheter and a Y-connector. The ADS is adapted to be placed in an abdomen such that abdominal movement, diaphragmatic movement, or a decrease in intracranial pressure causes CSF to flow from the ADS through the ventricular catheter and into a ventricle in the brain.

### BACKGROUND

### Field

Embodiments of the present invention generally relate to cerebrospinal fluid shunts and, in particular, to an improved apparatus for shunting cerebrospinal fluid that may be used to treat hydrocephalus.

### Description of the Related Art

In a normal mammal, cerebrospinal fluid (CSF) is created within ventricles of the brain and flows into and out from the spinal column. CSF flow is affected by a combination of forces acting upon fluids within the body including arterial pulsations, body movement, body orientation (i.e., standing, sitting, or prone), atmospheric pressure, respiration, diaphragmatic movement, abdominal motion, and the like. When the flow of CSF is constrained, a condition known as hydrocephalus occurs which results in CSF pressure increasing in the head. Untreated hydrocephalus may lead to brain damage and/or death.

Currently, hydrocephalus is treated with a CSF shunt that facilitates channeling CSF from the head or spine into another portion of the body (e.g., vein or peritoneal cavity) where the CSF is absorbed. A typical shunt comprises a ventricular catheter, a subgaleal catheter, a one-way pressure activated valve, and a distal subcutaneous peritoneal catheter. Some shunts also include a reservoir preceding the valve and an anti-siphon regulatory device after the valve. The ventricular catheter has its proximal end placed in a ventricle in the brain and a distal end that is connected to the reservoir or valve. The reservoir, valve and anti-siphon device may be connected in series and placed under the skin, typically, on the back or side of the skull. The peritoneal catheter has a proximal end that is connected to the anti-siphon device and a distal end extending to the site of CSF absorption (into a vein, the peritoneal cavity, or pleural cavity).

In operation, as CSF is produced in the ventricles and increases the pressure in the ventricles, the valve opens when the pressure exceeds the pressure threshold of the valve, the catheter resistance, the intraperitoneal pressure, plus the hydrostatic pressure difference. When the valve opens, CSF flows through the shunt to the peritoneal cavity or vein where the CSF is absorbed by the body. The reservoir is provided as a CSF access device and the anti-siphon device ensure that a siphon effect does not maintain the valve in on open position and drain all the CSF from the brain and spine (the reservoirs are in general only deformable by significant external pressure, they are otherwise non-compliant to physiological pressures).

A major problem with a shunt is that it is purely a pressure-based device that forms an alternate CSF path. Consequently, the typical shunt attenuates the other forces that effect CSF flow. Thus, the complex flow path for CSF is bypassed by the shunt and results in a variety of medical conditions such as intracranial hypotension and slit ventricles.

In addition, shunts routinely become obstructed and cease shunting CSF. One specific obstruction that routinely occurs is caused by the choroid plexus in the ventricle entering the open end of the ventricular catheter. Once clogged, brain surgery is required to replace the ventricular catheter.

Therefore, there is a need for apparatus for shunting CSF that more closely mimics the complex movement of CSF within the body and mitigates ventricular catheter obstruction.

### SUMMARY

Apparatus for shunting cerebrospinal fluid is provided substantially as shown in and/or described in connection with at least one of the figures, as set forth more completely in the claims.

Various features and advantages of the present disclosure may be appreciated from a review of the following detailed description of the present disclosure, along with the accompanying figures in which like reference numerals refer to like parts throughout.

### BRIEF DESCRIPTION OF THE DRAWINGS

So that the manner in which the various features of the present invention can be understood in detail, a particular description of the invention, may be had by reference to embodiments, some of which are illustrated in the appended drawings. It is to be noted, however, that the appended drawings illustrate only typical embodiments of this invention and are therefore not to be considered limiting of its scope, for the invention may admit to other equally effective embodiments.

FIG. 1 depicts a schematic of a person with a cerebrospinal fluid shunt having an improved ventricular catheter and/or an artificial dural sac in accordance with at least one embodiment of the invention;

FIG. 2 depicts a plan view of a sleeved ventricular catheter in accordance with at least one embodiment of the invention;

FIG. 3 depicts a cross sectional view of a connector reservoir of the shunt of FIG. 2 in accordance with at least one embodiment of the invention;

FIG. 4 depicts a long axis cross sectional view of a ventricular catheter of the shunt along line 4-4 in FIG. 2 in accordance with at least one embodiment of the invention;

FIG. 5 depicts a short axis cross sectional view of a ventricular catheter of the shunt along line 5-5 in FIG. 2 in accordance with at least one embodiment of the invention; and

FIG. 6 depicts a cross sectional view of an artificial dural sac of the shunt in FIG. 2 in accordance with at least one embodiment of the invention.

### DETAILED DESCRIPTION

Embodiments of the present invention comprise a cerebrospinal fluid (CSF) shunt having an improved ventricular catheter and/or an artificial dural sac. Such a shunt is improved over currently available shunts by including a sleeved ventricular catheter, an artificial dural sac or both. The sleeved ventricular catheter comprises an inner catheter having a plurality of holes in the sides of the catheter and a sleeve having a larger diameter than the inner catheter which circumscribes the inner catheter. The sleeve is coupled (for example, via ligature) to the inner catheter at a distance away from the inner catheter tip. The inner catheter comprises a plurality of holes near the distal end of the inner catheter. The sleeve defines a gap surrounding the inner catheter to allow CSF to flow in the gap to the holes in the inner catheter. As such, the sleeve protects the holes from becoming obstructed with brain matter, e.g., choroid plexus. The sleeved ventricular catheter may be used in a conventional shunt, e.g., connected to a reservoir (optional), one-way valve, anti-siphon device (optional) and a peritoneal catheter.

The shunt may further include, or alternatively include, an artificial dural sac (ADS). The ventricular catheter, whether standard or sleeved, is coupled to a 3-port connector reservoir where a first port is coupled to the coupling catheter (or subgaleal catheter), a second port is coupled to a one-way pressure valve (with siphon regulatory device (such as an anti-syphon device or Starling resistor) and a third port is coupled to a subcutaneous catheter that leads to the ADS located in the abdomen. When the ADS is used, the syphon regulatory device is necessary (not optional as in the embodiment without an ADS) to avoid having the ADS empty into the peritoneal cavity when the head is elevated. The ADS is located caudal to the level of the siphon regulatory device. In operation, the ADS and its catheter fill with a volume of CSF. The ADS is a flexible pressure responsive reservoir. When compressed, the ADS supplies CSF back to the ventricles in a more natural flow pattern, e.g., the ADS is compressed in response to increased intra-abdominal pressure, increase in hydrostatic pressure (a head down position), a decreasing intracranial pressure (as during inspiration) and the like.

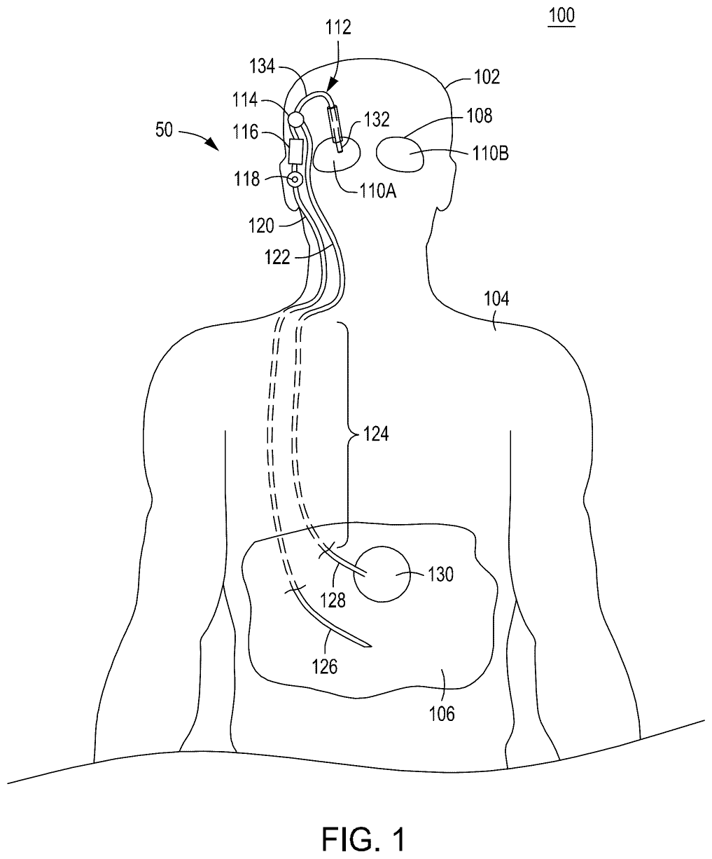

FIG. 1 depicts a schematic of a person 100 with a cerebrospinal fluid (CSF) shunt 50 having an improved ventricular catheter 112 and/or an artificial dural sac (ADS) 130 in accordance with at least one embodiment of the invention. The person 100 has a head 102 and torso 106. The head contains a brain 108 located in the head 102 and an abdomen 106 located in the torso 104. The shunt 50 extends from a ventricle 110A or 110B in the brain 102 to the abdomen 106 such that excess CSF can flow from the head 102 to the abdomen 106. In one embodiment, the shunt 50 comprises a ventricular catheter 112, a connector reservoir 114, a one-way valve 116, a siphon regulatory device 118, a subcutaneous peritoneal catheter 120, a subcutaneous ADS catheter 122 and an ADS 130. The improved shunt 50 may contain the sleeved ventricular catheter, the ADS or both.

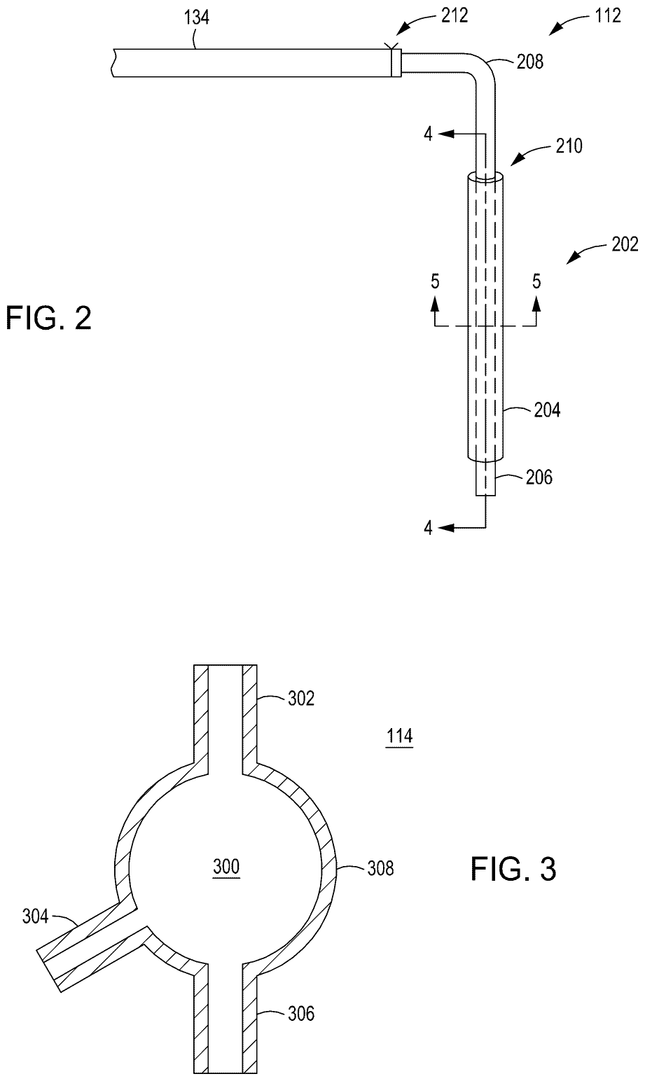

The ventricular catheter 112 has a proximal end 132 that is positioned in one of the ventricles 110A or 110B. The ventricular catheter 112 extends outside the skull though a burr hole and and is coupled to a coupling or subgaleal catheter 134. The coupling catheter 134 is routed subcutaneously to the connector reservoir 114. The ventricular catheter 112 is described in detail with respect to FIGS. 2, 4 and 5 below. If the embodiment contains an ADS, the reservoir forms a Y-connector that couples the coupling catheter 134 to the ADS catheter 122 and the one-way valve 116. The connector reservoir 114 is described in detail with respect to FIG. 3 below. The combination of the valve 116, an optional anti-siphon device 118 and the distal catheter 120 form a conventional shunt assembly that carries CSF from the valve 116 to portion 126 of the peritoneal catheter 120 located in the abdomen 106. The portion of the catheters 120 and 124 typically are located subcutaneously along the back of the head 102. A central portion 124 of the catheters 120 and 122 is shown as dashed and is routed along the chest of the person 100. The distal end 126 of the catheter 120 terminate in the peritoneal cavity of the abdomen 106. The distal end 128 of the ADS catheter 122 terminates in the ADS 130 located in the abdomen 106 (submuscular, extraperitoneal space).

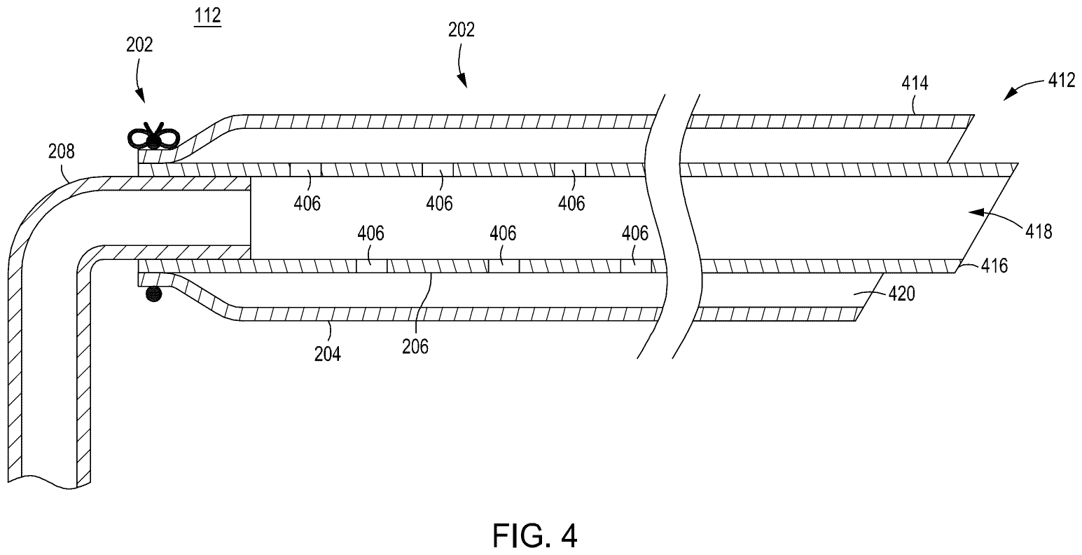

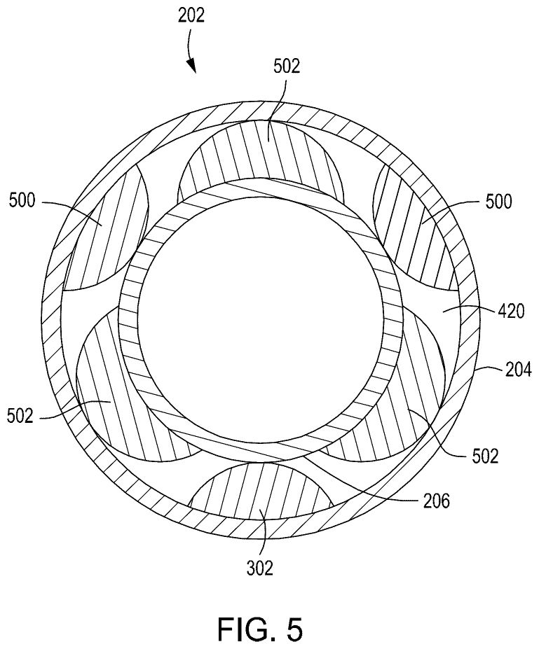

FIG. 2 depicts a plan view of the sleeved ventricular catheter 112 in accordance with at least one embodiment of the invention. In one embodiment, the sleeved ventricular catheter 112 comprising a coupling or subgaleal catheter 134, a coupler 208 (e.g., a 90 degree elbow coupler), and a sleeved catheter 202. In one embodiment, the coupler 208 is manufactured of plastic. The coupling catheter 200 may be attached to the coupler 208 using a ligature 212 formed from a tied suture. The sleeved catheter 202 may be attached to the coupler 208 using a ligature 210 located within the burr hole, outside the dura matter. As described in detail with respect to FIGS. 4 and 5 below, the sleeved catheter 202 comprises an inner catheter 206 and an outer catheter 204, where the outer catheter 204 forms a protective sleeve.

FIG. 3 depicts a cross sectional view of a connector reservoir 114 of the shunt 50 of FIG. 1 in accordance with at least one embodiment of the invention. The reservoir 114 forms a Y-coupler to attach the coupling catheter 200 to a first port 302, the shunt valve 116 to a second port 304 and the ADS catheter 120 to a third port 306. In one embodiment, the ports 302 and 306 have larger diameters than the diameter of port 304. For example, port 302 may have an inner diameter greater than about 1.8 mm, port 306 may have an inner diameter greater than about 1.8 mm and port 304 may have an inner diameter of about 1 mm. The low resistance pathway is bidirectional from the ventricular catheter to the ADS. The higher resistance pathway is a one-way pathway from the reservoir to the peritoneal cavity. The ports 302, 304, and 306 couple to a central reservoir 300 having a volume of about 2 ml. The reservoir 300 facilitates flow of CSF from the ventricular catheter to the valve and the ADS as well as CSF flow from the ADS to the ventricular catheter. Consequently, the CSF flow is bidirectional to and from the ADS via the reservoir 300. Reservoir 300 provides a percutaneous needle access to the CSF to test the fluid and to inject medication.

FIG. 4 depicts a cross sectional view of a portion of the ventricular catheter 112 of the shunt 50 along line 4-4 in FIG. 2 in accordance with at least one embodiment of the invention. The portion of ventricular catheter 112 comprises an coupler 208 coupled to a sleeved catheter 202 using a ligature 210. In the depicted embodiment, the coupler 208 is a 90-degree elbow. In other embodiments, the coupler 208 may be a cylinder without a bend or with other amounts of bend. The sleeved catheter 202 comprises an inner catheter 206 that is surrounded, at least in part, by an outer (sleeve) catheter 204 where the outer catheter 204 has a larger diameter than the diameter of the inner catheter 206. In one exemplary embodiment, the inner catheter 206 has an outer diameter that is less than the inner diameter of the outer catheter 204 by between 0.4 mm and 0.6 mm. In exemplary embodiments, one or both of the catheters 204 and/or 206 may have a beveled distal end 414 and/or 416. In some embodiments, the distal end 416 of the inner catheter 206 may extend beyond the distal end 414 of the outer catheter 204 (i.e., the outer catheter 204 extends along at least a portion of the inner catheter 206. The inner catheter 206 comprises a plurality of apertures 406 distributed along a distal portion of the inner catheter 206. These apertures 406 along with the end of the inner catheter 206 permits ingress and egress of CSF through the sleeved ventricular catheter 202. The apertures 406 are spaced far enough apart such that tissue cannot extend from one aperture to another (i.e., tether the catheter). The apertures 406 begin about 2 cm from the end 416 of the inner catheter 206.

Once placed in a ventricle, the sleeved catheter 202 facilitates CSF flow into and/or out of a distal end opening 418 and into a circumferential gap 420 between the inner catheter 206 and the outer catheter 204. In one embodiment, the outer catheter 204 is sealed to the inner catheter 206 at a junction with the elbow 208. The elbow 208 is manufactured of hard plastic such that a ligature 210 may be formed by tying a suture around the inner and outer catheters 206 and 204. The outer sleeve catheter 204 protects the apertures 406 in the inner cathode from being clogged with brain matter such as the choroid plexus.

FIG. 5 depicts a short axis cross sectional view of a ventricular catheter 202 of the shunt 50 along 5-5 in FIG. 1 in accordance with at least one embodiment of the invention. The sleeved ventricular catheter 202 comprises the outer catheter 204 circumscribing the inner catheter 206 and forming a gap 420 between the catheters 204 and 206. The gap 420 may be between 0.4 and 0.6 mm wide.

In one embodiment, the inner surface of the outer catheter 204 and the outer surface of the inner catheter 206 may be smooth (not shown). In other embodiments, the inner surface of the outer catheter 204 and the outer surface of the inner catheter 206 may have a plurality of longitudinal ribs 500/502 running the length of the catheters 204 and 206. In one embodiment there may be 3 ribs 500/502 on each surface. The ribs 500/502 may be positioned at offset 60 degree intervals and are intended to maintain the gap 420 between the catheters 204 and 206 to promote ingress and egress of CSF. In an exemplary embodiment, the outer catheter 204 has an inner diameter of about 3.2 mm, and an outer diameter of about 3.6 mm and an inner diameter of the rib 500 of about 2.7 mm, while the inner catheter 206 has an outer diameter of about 2.2 mm, an inner diameter of 1.8 mm, and an outer diameter of the rib 502 of about 2.7 mm.

Although the sleeved ventricular catheter 202 is shown as functioning with a shunt 50 having an ADS, it should be understood that the sleeved ventricular catheter 202 may be used in a conventional shunt assembly, e.g., the sleeved ventricular catheter 202 is coupled to the one way valve without the use of a Y-junction reservoir. In such an embodiment, the sleeved ventricular catheter 202 provides the benefit to the conventional shunt of having a reduced chance of becoming obstructed due to the protective nature of the sleeve and, if a ventricular revision is needed because both gap 420 and opening 418 become occluded, the catheter 202 will not be tethered inside the ventricle. An untethered catheter lessens the risk of bleeding with the removal of the catheter protected by the sleeve.

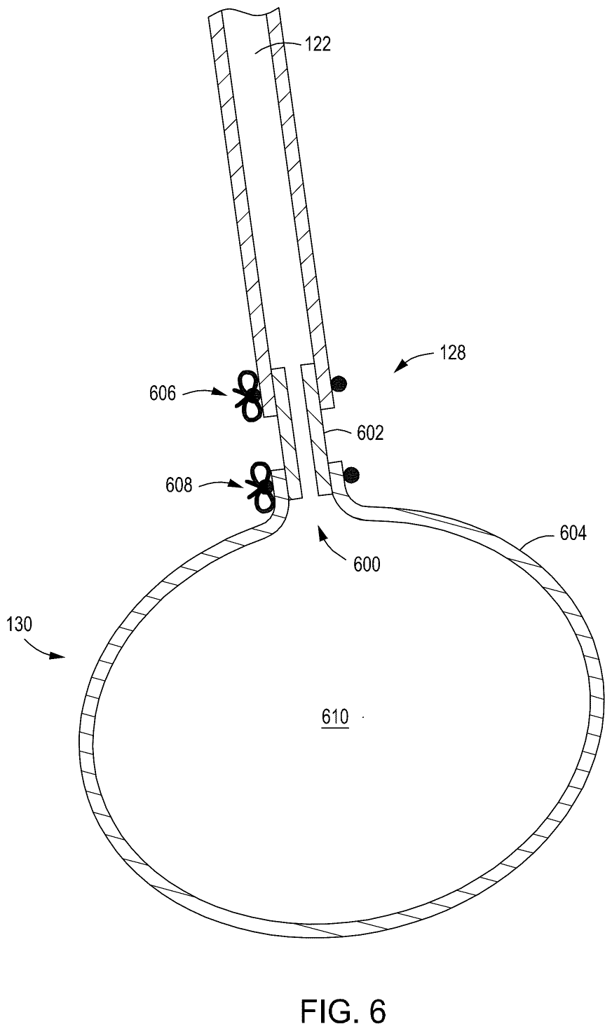

FIG. 6 depicts a cross sectional view of an artificial dural sac (ADS) 130 of the shunt 50 in FIG. 1 in accordance with at least one embodiment of the invention. The ADS 130 is coupled to the distal end 128 of the subcutaneous ADS catheter 122. A plastic cylindrical coupler 602 may be attached to the catheter 122 via, for example, a ligature 606 formed with a suture and is attached to the ADS 130 at a port 600 using a similar ligature 608. In other embodiments, the coupler may be manufactured as a portion of the ADS 130 and the catheter 122 may be attached directly to the ADS 130. The ADS 130 has a thin, flexible outer wall 604 (e.g., approximately 0.2 mm thick) defining a volume 610 where CSF collects. The ADS 130 may have the shape of a spherical balloon, ellipsoid, disc, rectangle, and the like. The full volume 610 of a small ADS may be about 5 ml and the full volume 610 of a large ADS may be about 100 ml. The full volume of a medium ADS may be about 50 ml. Once the ADS is full of CSF, body movement (e.g., abdominal muscular contraction, diaphragmatic motion, abdominal motion, etc.) will compress the ADS and cause CSF to flow into the ventricular catheter and into the ventricles in a more natural CSF flow path. The ADS may also supply CSF upon the occurrence of a decrease in intracranial pressure. An ADS plus large diameter ADS catheter and ventricular catheter may be used without a shunt to increase intracranial compliance. Conditions such as spinal stenosis, caraniocervical junctions CSF flow restrictions, some cases of normal pressure hydrocephalus, as well as cases of syringomyelia might benefit from placement of an ADS without a shunt (i.e., without a valve and its accompanying components shunting CSF to the peritoneal cavity).

To allow the ADS 130 to continue functioning as a child grows, additional catheter 122 is placed in the pre-peritoneal space such that the extra catheter extends as the child grows. Additionally, during initial shunt placement surgery, the ADS may be used as a reservoir for one or more antibiotics (such as gentamicin) using a prophylactic dose.

Here multiple examples have been given to illustrate various features and are not intended to be so limiting. Any one or more of the features may not be limited to the particular examples presented herein, regardless of any order, combination, or connections described. In fact, it should be understood that any combination of the features and/or elements described by way of example above are contemplated, including any variation or modification which is not enumerated, but capable of achieving the same. Unless otherwise stated, any one or more of the features may be combined in any order.

As above, figures are presented herein for illustrative purposes and are not meant to impose any structural limitations, unless otherwise specified. Various modifications to any of the structures shown in the figures are contemplated to be within the scope of the invention presented herein. The invention is not intended to be limited to any scope of claim language.

Where “coupling” or “connection” is used, unless otherwise specified, no limitation is implied that the coupling or connection be restricted to a direct physical coupling or connection.

Where conditional language is used, including, but not limited to, “can,” “could,” “may” or “might,” it should be understood that the associated features or elements are not required. As such, where conditional language is used, the elements and/or features should be understood as being optionally present in at least some examples, and not necessarily conditioned upon anything, unless otherwise specified.

Where lists are enumerated in the alternative or conjunctive (e.g., one or more of A, B, and/or C), unless stated otherwise, it is understood to include one or more of each element, including any one or more combinations of any number of the enumerated elements (e.g. A, AB, AC, ABC, ABB, etc.). When “and/or” is used, it should be understood that the elements may be joined in the alternative or conjunctive.

While the foregoing is directed to embodiments of the present invention, other and further embodiments of the invention may be devised without departing from the basic scope thereof, and the scope thereof is determined by the claims that follow.

1. Apparatus for shunting cerebrospinal fluid (CSF) comprising:
a ventricular catheter having a proximal end adapted to be positioned in a ventricle of a brain;
a Y-coupler having a first port coupled to the ventricular catheter, a second port coupled to a one-way valve, siphon regulatory device, and a peritoneal catheter, and a third port coupled to an artificial dural sac (ADS) catheter; and
an ADS coupled to the ADS catheter.

2. The apparatus of claim 1 wherein the ADS comprises a flexible wall defining a volume adapted for containing CSF.

3. The apparatus of claim 2 wherein the ADS is adapted for placement in an abdomen and, upon being compressed by abdominal movement, diaphragmatic movement or a decrease in intracranial pressure, CSF is caused to flow through the ventricular catheter into the ventricle.

4. The apparatus of claim 1 wherein the ventricular catheter comprises an inner catheter and an outer catheter which forms a sleeve circumscribing at least a portion of the inner catheter.

5. The apparatus of claim 4 wherein the outer diameter of the inner catheter and the inner diameter of the outer catheter form a gap of about 0.4 to 0.6 mm.

6. The apparatus of claim 4 wherein the inner catheter comprises a plurality of apertures that are protected by the outer catheter forming a sleeve.

7. The apparatus of claim 1 wherein an outer surface of the inner catheter comprises a plurality of longitudinal ribs and an inner surface of the outer catheter comprise a plurality of longitudinal ribs.

8. The apparatus of claim 1 wherein the ADS is located caudal to the siphon regulatory device.

9. Apparatus for shunting cerebrospinal fluid (CSF) comprising:
a ventricular catheter having a proximal end adapted to be positioned in a ventricle of a brain and comprising an inner catheter and an outer catheter which forms a sleeve circumscribing at least a portion of the inner catheter;
a one-way valve coupled to the ventricular catheter; and
a peritoneal catheter coupled to the one-way valve.

10. The apparatus of claim 9 wherein the outer diameter of the inner catheter and the inner diameter of the outer catheter form a gap of about 0.4 to 0.6 mm.

11. The apparatus of claim 9 wherein the inner catheter comprises a plurality of apertures that are protected by the outer catheter forming a sleeve.

12. The apparatus of claim 9 wherein an outer surface of the inner catheter comprises a plurality of longitudinal ribs and an inner surface of the outer catheter comprise a plurality of longitudinal ribs.

13. The apparatus of claim 9 further comprising:
a Y-coupler having a first port coupled to the ventricular catheter, a second port coupled to the one-way valve and a siphon regulatory device,, and a third port coupled to an artificial dural sac (ADS) catheter; and
an ADS coupled to the ADS catheter.

14. The apparatus of claim 13 wherein the ADS comprises a flexible wall defining a volume adapted for containing CSF.

15. The apparatus of claim 14 wherein the ADS is adapted for placement in an abdomen and, upon being compressed by abdominal movement, diaphragmatic movement, or a decrease in intracranial pressure, CSF in the ADS flows through the ventricular catheter into the ventricle.

16. The apparatus of claim 13 wherein the ADS is located caudal to the siphon regulatory device.

17. Apparatus for shunting cerebrospinal fluid (CSF) comprising:
a ventricular catheter having a proximal end adapted to be positioned in a ventricle of a brain and comprising an inner catheter and an outer catheter which forms a sleeve circumscribing at least a portion of the inner catheter;
a Y-coupler having a first port coupled to the ventricular catheter, a second port coupled to a one-way valve, a siphon regulatory device and a peritoneal catheter, and a third port coupled to an artificial dural sac (ADS) catheter; and
an ADS coupled to the ADS catheter.

18. The apparatus of claim 17 wherein the ADS comprises a flexible wall defining a volume adapted for containing CSF.

19. The apparatus of claim 18 wherein the ADS is adapted for placement in an abdomen and, upon being compressed by abdominal movement, diaphragmatic movement or a decrease in intracranial pressure, CSF in the ADS flows through the ventricular catheter into the ventricle.

20. The apparatus of claim 17 wherein the outer diameter of the inner catheter and the inner diameter of the outer catheter form a gap of about 0.4 to 0.6 mm.

21. The apparatus of claim 17 wherein the inner catheter comprises a plurality of apertures that are protected by the outer catheter forming a sleeve.

22. The apparatus of claim 17 wherein an outer surface of the inner catheter comprises a plurality of longitudinal ribs and an inner surface of the outer catheter comprise a plurality of longitudinal ribs.

23. The apparatus of claim 17 wherein the ADS is located caudal to the siphon regulatory device.

24. Apparatus for storing cerebrospinal fluid (CSF) comprising:
a ventricular catheter having a proximal end adapted to be positioned in a ventricle of a brain; and
an ADS coupled to a distal end of the ventricular catheter.

25. The apparatus of claim 24 wherein the ADS comprises a flexible wall defining a volume adapted for containing CSF.

26. The apparatus of claim 25 wherein the ADS is adapted for placement in an abdomen and, upon being compressed by abdominal movement, diaphragmatic movement or a decrease in intracranial pressure, CSF is caused to flow through the ventricular catheter into the ventricle.

27. The apparatus of claim 24 wherein the ventricular catheter comprises an inner catheter and an outer catheter which forms a sleeve circumscribing at least a portion of the inner catheter.

28. The apparatus of claim 27 wherein the outer diameter of the inner catheter and the inner diameter of the outer catheter form a gap of about 0.4 to 0.6 mm.

29. The apparatus of claim 27 wherein the inner catheter comprises a plurality of apertures that are protected by the outer catheter forming a sleeve.

30. The apparatus of claim 24 wherein an outer surface of the inner catheter comprises a plurality of longitudinal ribs and an inner surface of the outer catheter comprises a plurality of longitudinal ribs.
